(19) United States
(12) Patent Application Publication
(10) Pub. No.: US 2008/0021193 A1
(43) Pub. Date: Jan. 24, 2008

(54) Process of manufacturing DOPO derivatives for printed circuit board and low-earth orbit spacecraft applications

(75) Inventors: Ching-Hsuan Lin, Taichung City (TW); Chun-Hung Lin, Fongshan City (TW)

Correspondence Address: BIRCH STEWART KOLASCH & BIRCH, PO BOX 747, FALLS CHURCH, VA, 22040-0747, US

(73) Assignee: NATIONAL CHUNGHSING UNIVERSITY

(21) Appl. No.: 11/819,617

(22) Filed: Jun. 28, 2007

(30) Foreign Application Priority Data
Jun. 29, 2006 (TW) 95123635

(51) Int. Cl. C07D 521/00; C08G 63/44

(52) U.S. Cl. 528/363; 549/388

(57) ABSTRACT

A method of manufacturing new materials for a printed circuit board and Low-Earth Orbit (LEO) spacecraft is provided. The present invention includes dinitro, diamine, various phosphorous-containing polyimides and polyamides, and synthesizing methods thereof. The polymers of the embodiment of present invention exhibit good flame retardancy, high glass transition temperature, good mechanical properties and superior oxygen resistance, so they are good materials for Low-Earth Orbit applications. Besides, these polymers can also be used as matrix for halogen-free flexible printed circuit board.

### RELATED APPLICATIONS

The application claims priority to Taiwan Application Serial Number 95123635, filed Jun. 29, 2006, which is herein incorporated by reference.

### BACKGROUND

1. Field of Invention

The present invention relates to a process of manufacturing DOPO (9,10-dihydro-9-oxa-10-phosphaphenanthrene 10-oxide) derivatives for printed circuit board and Low-Earth Orbit spacecraft applications. More particularly, the present invention relates to the process of manufacturing DOPO derivatives of phosphorous-containing polyimides and polyamides.

2. Description of Related Art

Organic and polymeric materials used in orbiting satellite are eroded by atomic oxygen (AO), ultraviolet (UV) rays, and vacuum ultraviolet (VUV) irradiations at a Low Earth Orbit (LEO) altitude of 160˜800 Km. Although the AO were reduced in high Earth Orbit (HEO), the UV, VUV, electron-proton, and corpuscular radiation still influence the organic and polymeric materials of the orbiting satellite. These irradiation energies accumulated on the polymeric materials can break the bonds of the polymers and change the physical, mechanical and optical characters of the polymers.

There is no existing commercial polymeric material that can resist the erosion from an AO attack. The polymeric material in Low Earth Orbit requires coating of inorganic layers, such as aluminum oxide, silica, or chromium oxide layers, with a thickness of 500˜2000 Å to protect the polymeric material of the apparatus. However, inorganic layers cannot provide uniform protection against erosion, so the polymeric material may crack at the defect of the inorganic layer and breaking the bonding of the polymer. Moreover, when the environmental temperature changes, the different thermal expansion coefficients between the organic material and inorganic material can also cause the inorganic layer to break. Therefore, an AO resistible polymeric material is preferred for spacecraft application.

Research and development of the AO resistible polymeric materials now focuses on adding PPO (phenyl phosphine oxide) group to aromatic polymer (Polymer 1995, 36, 5-11; Polymer 1995, 36, 13-19; High Perform. Polym. 2001, 13, 23-34). Many research reports show polyphosphate formed on a polymer surface is effective in inhibiting AO erosion. The X-ray photoelectron spectroscopy (XPS) analysis has proved that the polyphosphate-containing polymer contains a high quantity of phosphorous and oxygen so as to provide AO-resistance capabilities for the polymeric material. The AO-resistance capabilities of these phosphorous-containing polyethers are higher than a commercial product “Kapton®” produced by Du Pond corporation.

Compared with “Kapton®”, the polymer without PPO group has poor AO-resistance capabilities. In contrast, the polymer with PPO group has AO-resistance 40 to 145 times higher than the “Kapton®”. In 1998, a polyether ketone film was published and tested on the Atlantis space shuttle; the result shows that interaction of the organic phosphorous and atomic oxygen could form a polyphosphate protection layer. The film with the polyphosphate protection layer has increased in reliability as compared with the inorganic polymeric material.

In general, aromatic polyimide with a high molecular weight provides good tenacity, flexibility, high glass transition temperature, solvent-tolerance, and high thermal stability. However, the disadvantage of the aromatic polyimide is that the yellow to amber color of the polyimide film cause high sunbeam absorbency. The formation of a charge-transfer complex (CTC) would darken the polyimide film, and the aromatic diamine with large functional group can be used to reduce the formation of the CTC in polyimide film. A polyimide with PPO group (J. Appl. Polym. Sci. 1983, 28, 2805-2812) was synthesized in 1983 to try to reduce the formation of the CTC; however, the added PPO group is not big enough so the formation of the CTC cannot be effectively prevented. In 2001, a diamine with PPO group (High Perform. Polym. 2001, 13, 23-34) was synthesized and applied to synthesize a polyimide with phosphorous-containing main chain by reacting the polyimide with various dianhydrides. The polyimide with phosphorous-containing main chain provides AO-resistance, UV-resistance and low sunbeam absorbency. However, the material is highly brittle and poor in mechanical character. In 2002, another polyimide with phosphorous-containing side chain (Macromolecules 2002, 35, 4968-4974) was synthesized. The polyimide with phosphorous-containing side chain can prevent the formation of CTC effectively to form a polyimide film with light color. Most of the polyimides with phosphorous-containing side chain provide excellent AO-resistance, mechanical characters (such as good tenacity), and 212° C.˜215° C. of glass transition temperature. It is therefore the polyimides with phosphorous-containing side chain that are applied to Low-Earth Orbit spacecraft applications.

Flexible print circuit (FPC) boards are classed as double layer FPC and three layer FPC. The research and development of the three layer FPC is focused on the adherence layer between the Kapton and copper foil, and the double layer FPC is focuses on synthesizing a soluble polyimide that can be applied to the copper foil. The common FPC is a three layer structure consisting of plasma-modified polyimide, Kapton, epoxy resin (adhesive), and copper foil. Because the thermal properties of the epoxy resin are poorer than the Kapton, the epoxy resin (adhesive) determines the thermal properties of the FPC. For the forgoing reasons, the current trend is towards developing double layer FPC with flame retardant and solvent-soluble polyimide.

### SUMMARY

The present invention is directed to a process of manufacturing DOPO derivatives for a printed circuit board and Low-Earth Orbit (LEO) spacecraft applications.

In accordance with the foregoing and other objectives of the present invention, the process of manufacturing phosphorous-containing polyimides is disclosed. A phosphorous-containing aromatic dinitro-compound, DOPOBQ-NB, was prepared by reacting a DOPOBQ with p-halo nitrobenzene (such as 1-fluoro-4-nitrobenzene). An exemplary synthesis strategy of the DOPOBQ-NB is shown in the following formula:

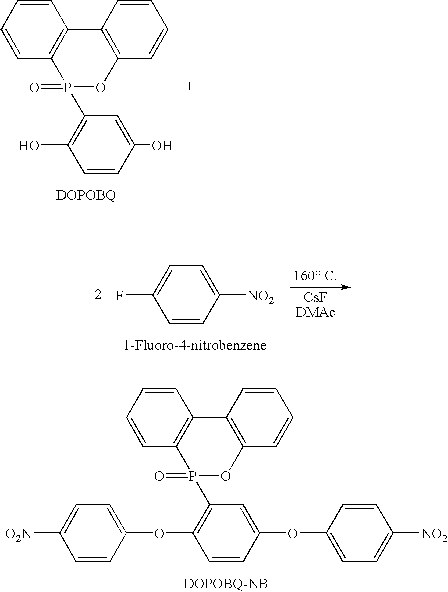

Another phosphorous-containing aromatic dinitro-compound, DOPONQ-NB, was prepared according to the preparation procedures of DOPOBQ-NB with the exception that the DOPONQ was substituted for the DOPOBQ. An exemplary synthesis strategy of the DOPONQ-NB is shown in the following formula:

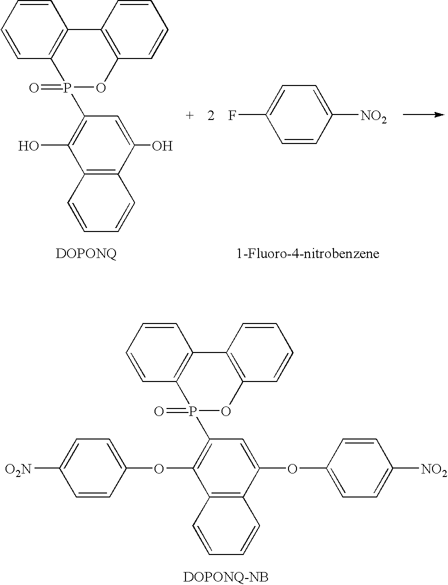

In accordance with an embodiment of the present invention, reacting the DOPOBQ-NB with a substituent group containing p-halo nitrobenzene to synthesize a substitute group-containing DOPOBQ-NB has a general formula represented by the following formula:

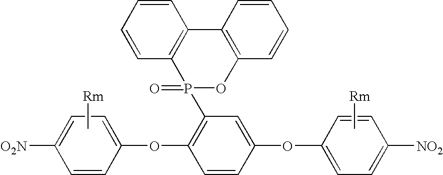

In accordance with another embodiment of the present invention, reacting the DOPONQ-NB with a substituent group containing p-halo nitrobenzene to synthesize a substitute group-containing DOPONQ-NB has a general formula represented by the following formula:

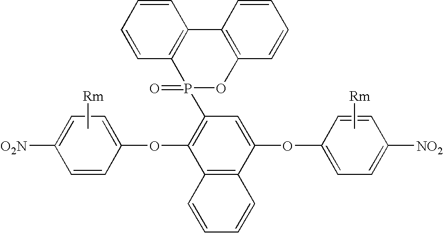

In accordance with the foregoing and other objectives of the present invention, the process of manufacturing DOPO derived diamines is disclosed. A DOPO derived diamine, DOPOBQ-AB, was prepared by reacting a DOPOBQ with hydrogen. An exemplary synthesis strategy of the DOPOBQ-AB is shown in the following formula:

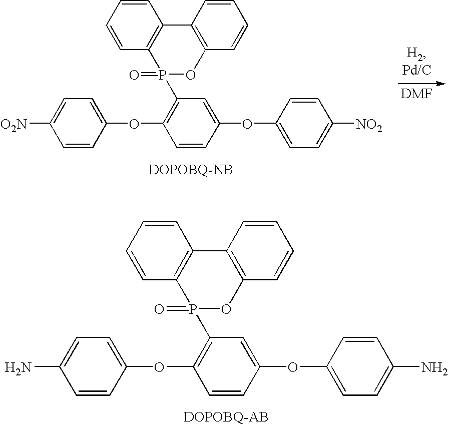

Another DOPO derived diamine, DOPONQ-AB, was prepared on the same procedures above-mentioned with the exception that the DOPONQ was substituted for the DOPOBQ. An exemplary synthesis strategy of the DOPONQ-AB is shown in the following formula:

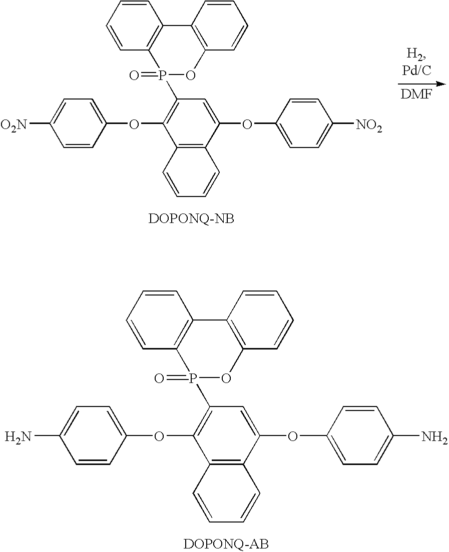

In accordance with an embodiment of the present invention, reacting the DOPOBQ-AB with a substituent group containing p-halo nitrobenzene to synthesize a substitute group-containing DOPOBQ-NB has a general formula represented by the following formula:

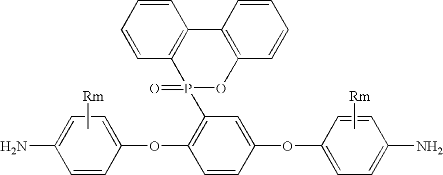

In accordance with another embodiment of the present invention, reacting the DOPONQ-AB with a substituent group containing p-halo nitrobenzene to synthesize a substitute group-containing DOPONQ-AB has a general formula represented by the following formula:

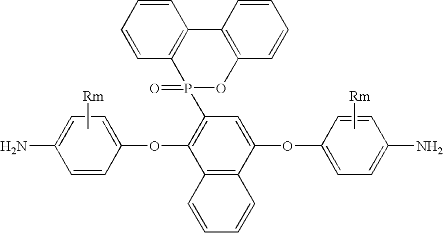

In accordance with the foregoing and other objectives of the present invention, the process of manufacturing phosphorous-containing polyimides is disclosed. Various DOPO derived phosphorous-containing polyimides are prepared by reacting a DOPOBQ-AB (or DOPONQ-AB) with series of dianhydrides. An exemplary synthesis strategy of the DOPO derived phosphorous-containing polyimides is shown in the following formula:

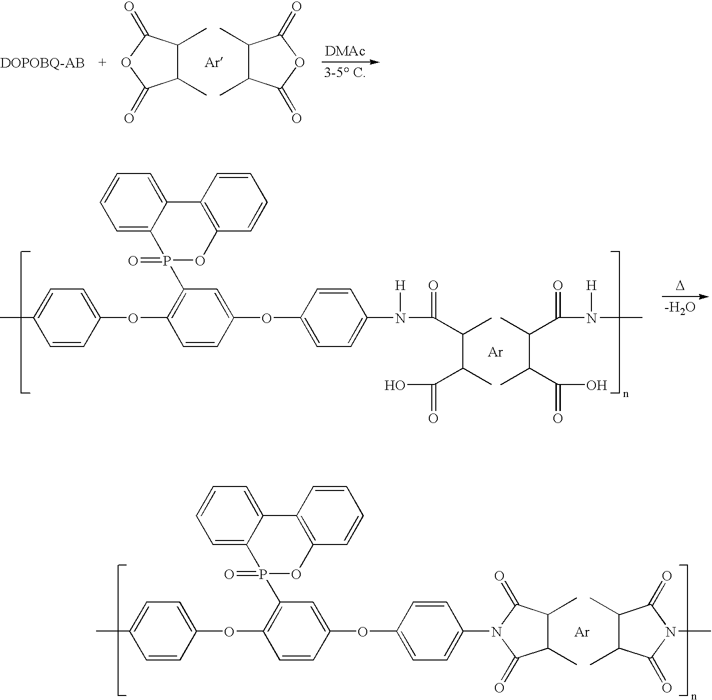

In accordance with embodiments of the present invention, the “Ar′” of dianhydrides, (a) PMDA, (b) BTDA, (c) OPDA, (d) BPDA, (e) 6FDA, and (f) BPADA, are presented as follow:

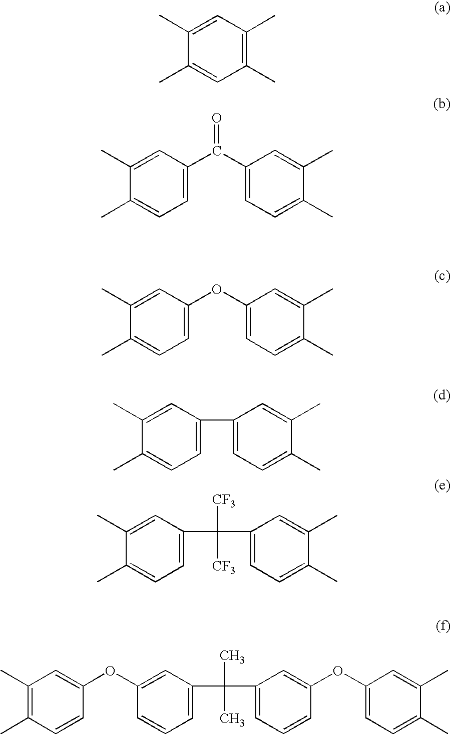

In accordance with the molecular weight analysis and the solubility analysis of the DOPO derived phosphorous-containing polyimides, introducing the DOPD group into the polyimide increases the solubility of the polyimides. The method of synthesis of the phosphorous-containing polyimides of the embodiments of the present invention are applied to form the dissoluble polyimide.

The phosphorous-containing thermoforming materials, DOPO derived polyimides, provide good mechanical properties such as a higher decomposition temperature than the phosphorous-containing polymers with P═O group in the main chain. Furthermore, the DOPO derived polyimides of the embodiment of the present invention have excellent penetrability with a cutoff wavelength within 342 nm ˜404 nm. The phosphorous-containing polyimides have less weight loss (%) in oxygen plasma destruction situation, and a poly(phosphate ester) can be formed by reacting the organic phosphorous with atomic oxygen to resist erosion from atomic oxygen attack. Therefore, the phosphorous-containing polyimides of the embodiments of the present invention provide the atomic oxygen resistance for Low-Earth orbit spacecraft applications.

In accordance with the foregoing and other objectives of the present invention, the process of manufacturing DOPO derived phosphorous-containing polyamides is disclosed. Various DOPO derived phosphorous-containing polyamides are prepared by reacting a DOPOBQ-AB (or DOPONQ-AB) with various diacids. An exemplary synthesis strategy of the phosphorous-containing polyamides is shown in the following formula:

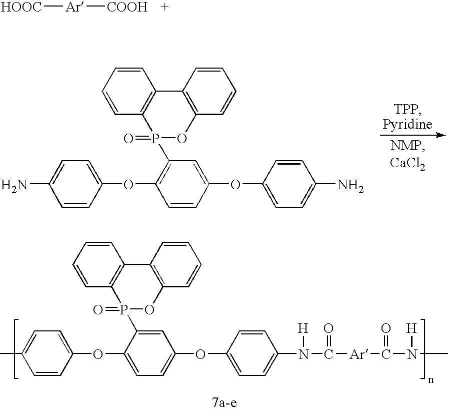

In accordance with embodiments of the present invention, the “Ar′” of diacids (a) to (e) are presented as follow:

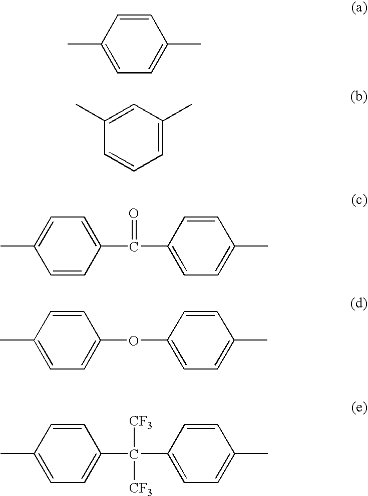

In accordance with the results of the different thermal analysis and the thermogravimetry analysis, the phosphorous-containing polyamides exhibit high glass transition temperature (Tg) and high decomposition temperature.

### BRIEF DESCRIPTION OF THE DRAWINGS

These and other features, aspects, and advantages of the present invention will become better understood with regard to the following description, appended claims, and accompanying drawings where:

FIG. 1 is a diagram of ¹H NMR (600 MHz) spectrum of DOPOBQ-NB in DMSO-D6 solution;

FIG. 2 is a diagram of ¹H NMR (600 MHz) spectrum of DOPOBQ-AB in DMSO-D6 solution; and

FIG. 3 is a diagram of weight loss (%) of the polyimides (5a˜5f) in oxygen plasma destruction.

### DESCRIPTION OF THE PREFERRED EMBODIMENTS

Organic phosphorous is a potential material for making halogen-free and flame-retardant FPC board because a polyphosphate protection layer can be formed by flaming the organic phosphorous. Moreover, the polyphosphate protection layer, formed by interacting the organic phosphorous and atomic oxygen, provides AO-resistance for LEO spacecraft applications. The embodiments of the present invention disclosed a series of polyimides to provide the multipurpose polymeric materials.

### Preparation Example 1

A phosphorous-containing aromatic dinitro-compound, DOPOBQ-NB, was prepared by reacting a DOPOBQ with p-halo nitrobenzene (such as 1-fluoro-4-nitrobenzene). An exemplary synthesis strategy of the DOPOBQ-NB is shown in the following formula:

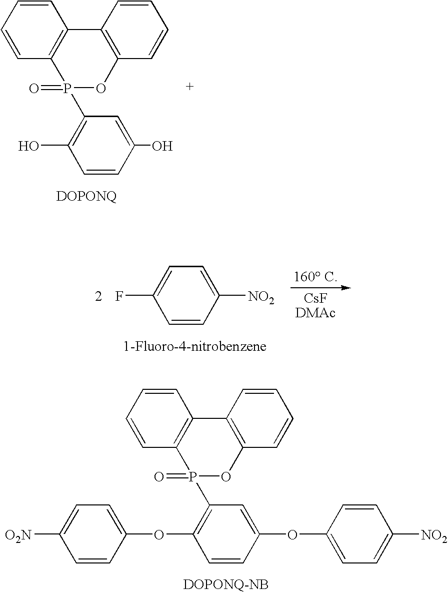

In accordance with an embodiment of the present invention, the synthesis of the DOPOBQ-NB was accomplished using the DOPOBQ monomer p-halo nitrobenzene as initiators in a solvent in the presence of a catalyst.

In the beginning, 28.5358 g (0.88 mole) of DOPOBQ, 25.0819 g (0.1778 mole) of 1-fluoro-4-nitrobenzene, 28.0711 g (0.1848 mole) of cesium fluoride (CsF) and 225.28 g N,N-dimethylacetamide (DMAc) were placed in a 500 ml flask. According to embodiments of the present invention, the reactant p-halo nitrobenzene can be 1-fluoro-4-nitrobenzene, 1-chloro-4-nitrobenzene, 1-bromo-4-nitrobenzene, or 1-iodo-4-nitrobenzene. The catalyst can be a compound composed of the elements of groups I A and VII A, such as CsF, KF, CsCl, or KCl; or inorganic alkali such as K₂CO₃, Na₂CO₃, KOH, or NaOH.

The reaction were carried out by stirring the reactants at 160° C. for 10 hours. Then the reaction temperature was cooled down to room temperature and the salts were filtered out to collect the filtrate. The filtrate was titrated to a 450 ml ethanol/water mixture (ethanol: water=½) with stirring, and a primrose yellow educt, DOPOBQ-NB, is separated out. The educt were further precipitated and filtered, and the filtered solid educt were washed by de-ion water. The solid educt were dried in a vacuum oven at 120° C. to obtain the raw DOPOBQ-NB and the yield was 55.55%. The raw DOPOBQ-NB was then re-crystallized with acetic anhydride to obtain DOPOBQ-NB solid with high purity. The yield of the DOPOBQ-NB was 62.97%, and the melting point was 252° C.

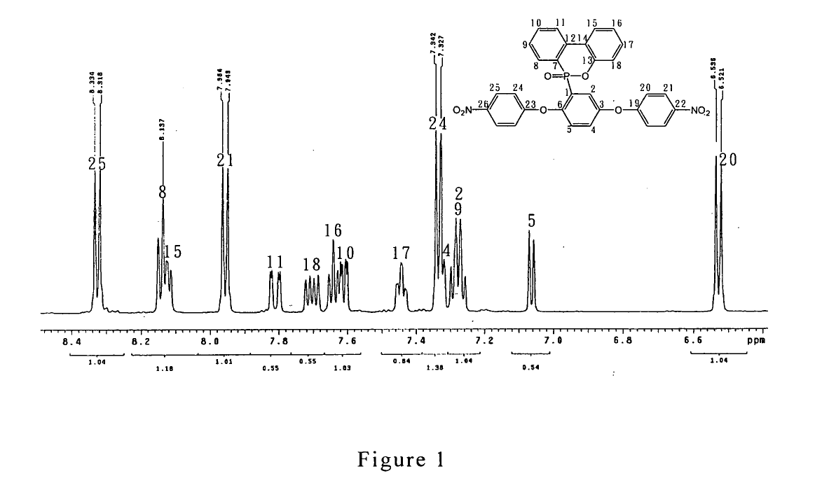

Referring to the FIG. 1. FIG. 1 is a diagram of ¹H NMR (600 MHz) spectrum of DOPOBQ-NB in DMSO-D6 solution. The ¹H NMR (600 MHz) spectrum proves the sample is the DOPOBQ-NB. In addition, a ³¹P NMR (300 MHz) spectrum also exhibits a high purity DOPOBQ-NB signal at 22.28 ppm without any byproduct.

In accordance with another embodiment of the present invention, a phosphorous-containing aromatic dinitro-compound, DOPONQ-NB, was prepared using the same principle by replacing the benzene ring of the DOPOBQ with naphthalene rings. That is, the synthesis of the DOPONQ-NB was accomplished using the DOPONQ monomer and p-halo nitrobenzene as initiators in a solvent in the presence of a catalyst.

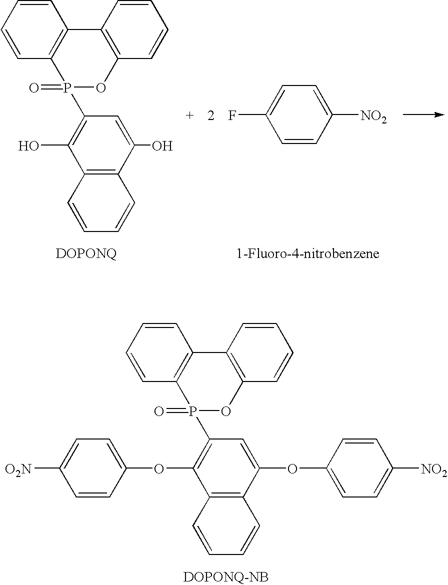

In accordance with the embodiments of the present invention, the reactant p-halo nitrobenzene can be 1-fluoro-4-nitrobenzene, 1-chloro-4-nitrobenzene, 1-bromo-4-nitrobenzene, or 1-iodo-4-nitrobenzene. The catalyst can be a compound composed of the elements of groups I A and VII A, such as CsF, KF, CsCl, or KCl; or inorganic alkali such as K₂CO₃, Na₂CO₃, KOH, or NaOH.

In accordance with an embodiment of the present invention, reacting the DOPOBQ-NB with a substituent group containing p-halo nitrobenzene to synthesize a substitute group-containing DOPOBQ-NB. An exemplary synthesis strategy of the substitute group-containing DOPOBQ-NB is shown in the following formula:

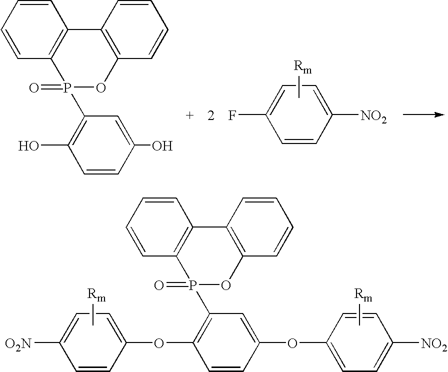

In accordance with another embodiment of the present invention, reacting the DOPONQ-NB with a substituent group containing p-halo nitrobenzene to synthesize a substitute group-containing DOPONQ-NB. An exemplary synthesis strategy of the substitute group-containing DOPONQ-NB is shown in the following formula:

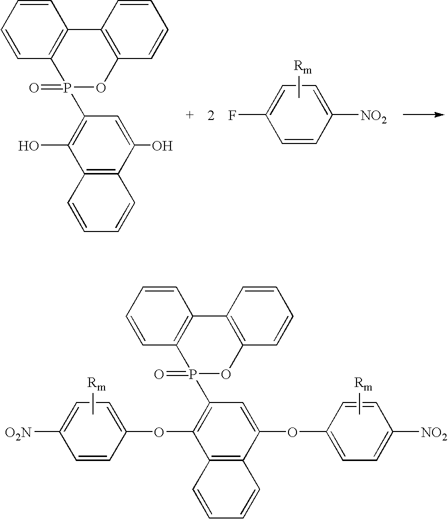

The “R” comprises hydrogen, —CH₃, —C₆H₅, or —CF₃. The “m” is an integer of 1˜2.

### Preparation Example 2

A DOPO derived diamine, DOPOBQ-AB, was prepared by reacting the DOPOBQ-NB with hydrogen to accomplish a catalytic hydrogenation. An exemplary synthesis strategy of the DOPOBQ-AB is shown in the following formula:

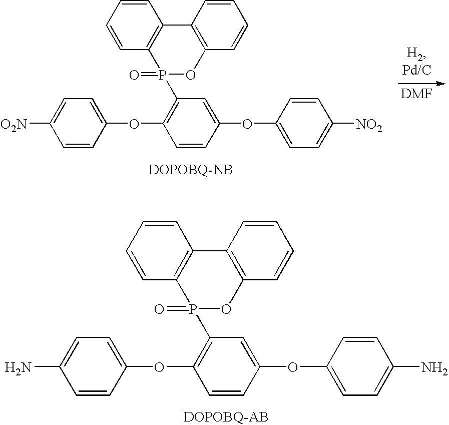

In accordance with an embodiment of the present invention, the synthesis of the DOPOBQ-AB was accomplished using the DOPOBQ-NB monomer as an initiator and hydrogen as a reactant in a solvent N,N-dimethylformamide (DMF) in the presence of a catalyst Pd/C. In the beginning of the synthesis of the DOPOBQ-AB, 6 g of DOPOBQ-NB, 0.1 g of Pd/C, and 50 g DMF were stirred in a 50 ml glass reactor. Nitrogen was introduced into the glass reactor and then bled from the glass reactor, and the operation was repeated at least three times. The reaction pressure was kept at 3.5 kg/cm² for 24 hours.

The Pd/C was filtered out after the reaction was accomplished, and the remainders were titrated to 500 ml water to precipitate the product. The above-mentioned operation was repeated twice. The educt was dried in a vacuum oven at 120° C. to obtain the raw DOPOBQ-AB and the yield was 93.53%. The raw DOPOBQ-AB was then re-crystallized by methanol to obtain high purity DOPOBQ-AB. The yield of the DOPOBQ-NB was 74.46%, and the melting point of the DOPOBQ-NB was 200° C.

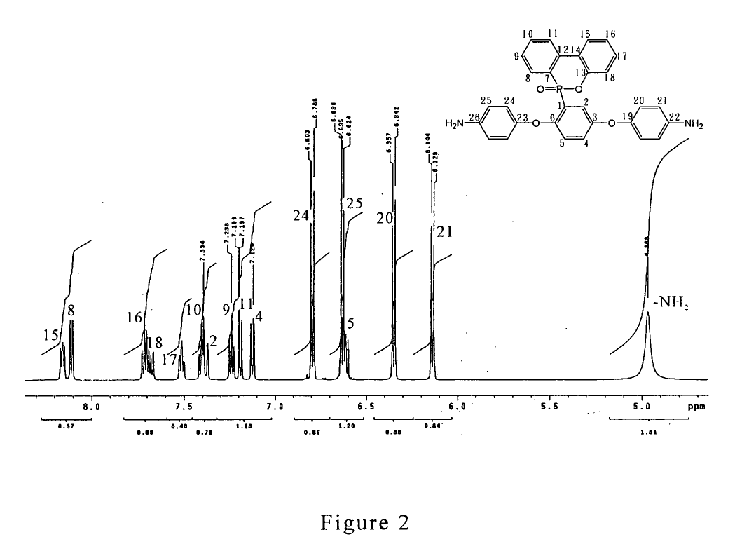

Referring to the FIG. 2. FIG. 2 is a diagram of ¹H NMR (600 MHz) spectrum of DOPOBQ-AB in DMSO-D6 solution. The ¹H NMR (600 MHz) spectrum shows the sample is the DOPOBQ-AB. In addition, a ³¹P NMR (300 MHz) spectrum also exhibits a high purity DOPOBQ-NB signal at 24.93 ppm without any byproducts.

In accordance with an embodiment of the present invention, another DOPO derived diamine, substituent group containing DOPOBQ-AB, was prepared on the same principle by reacting the substituent group containing DOPOBQ-NB with hydrogen to accomplish a catalytic hydrogenation. An exemplary synthesis strategy of the substituent group containing DOPOBQ-AB is shown in the following formula:

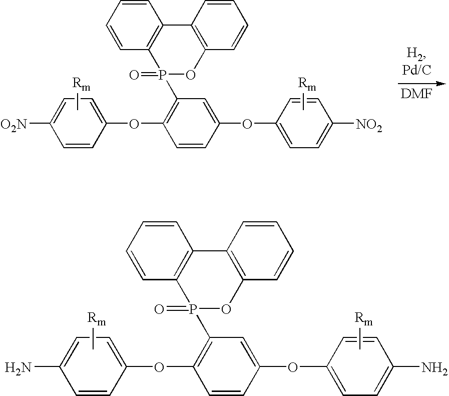

The “R” comprises hydrogen, —CH₃, —C₆H₅, or —CF₃. The “m” is an integer of 1˜2.

In accordance with an embodiment of the present invention, a DOPONQ-AB was prepared by replacing the DOPOBQ with DOPONQ to accomplish the same procedures as DOPOBQ-AB synthesis. An exemplary synthesis strategy of the DOPONQ-AB is shown in the following formula:

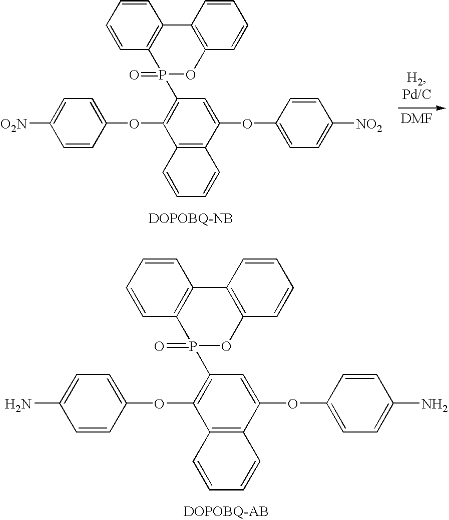

In accordance with another embodiment of the present invention, a substituent group containing DOPONQ-AB was prepared on the same principle by reacting the substituent group containing DOPOBQ-NB with hydrogen to accomplish a catalytic hydrogenation. An exemplary synthesis strategy of the substituent group containing DOPONQ-AB is shown in the following formula:

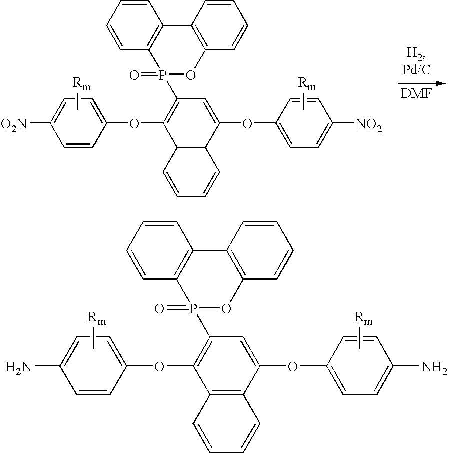

The “R” comprises hydrogen, —CH₃, —C₆H₅, or —CF₃. The “m” is an integer of 1˜2.

### Preparation Example 3

Phosphorous-containing polyimides were prepared by reacting a DOPOBQ-AB with series of dianhydrides. The phosphorous-containing polyimides have a general formula as follows:

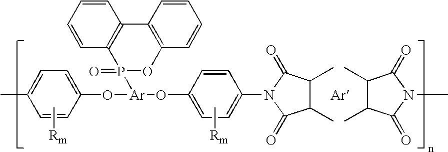

The “R” comprises hydrogen, —CH₃, —C₆H₅, or —CF₃. The “m” is an integer of 1˜2. The “Ar” is selected from the group consisting of following formulas:

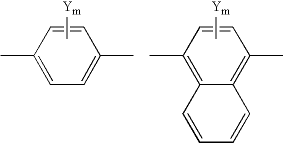

The “Y” comprises hydrogen, and C₁˜C₆ alkane. The “m” is an integer of 1˜2.

An exemplary synthesis strategy of the DOPO derived phosphorous-containing polyimides is shown in the following formula:

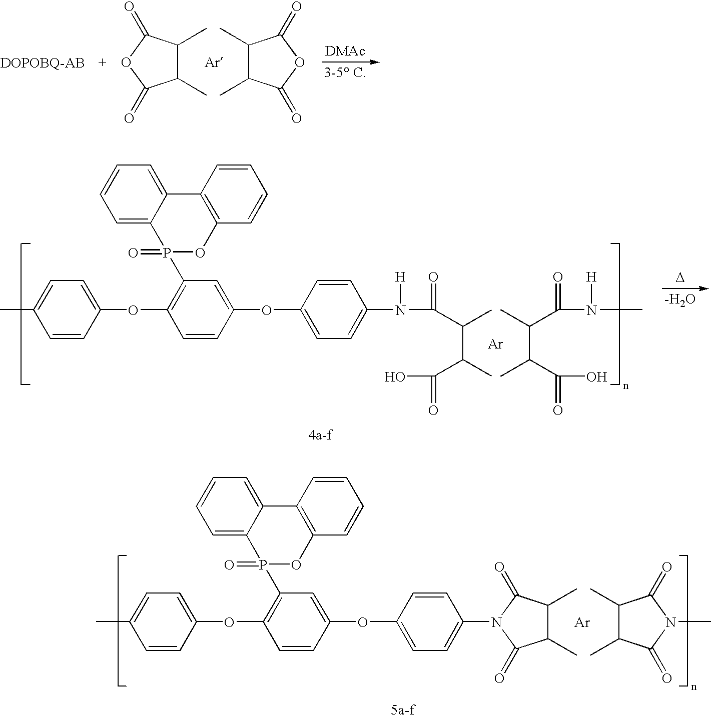

In accordance with embodiments of the present invention, the “Ar′” of dianhydrides can be (a) PMDA, (b) BTDA, (c) OPDA, (d) BPDA, (e) 6FDA, or (f) BPADA, are presented as follow:

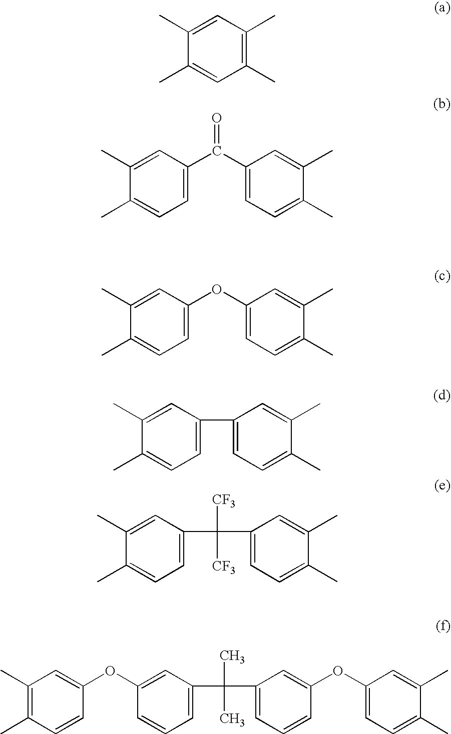

The synthesis of the DOPO derived phosphorous-containing polyimides (5a˜5f) may be accomplished by reacting the DOPOBQ-AB monomer with various dianhydrides (a˜f) in a similar manner, an exemplary preparation process is stated in the following description. 1.0130 g (2 mmole) of DOPOBQ-AB and 5.8219 g DMAc were stirred in a 100 ml 3-neck flask, and nitrogen was introduced into the 3-neck flask for 30 minutes. After the DOPOBQ-AB was dissolved in DMAc, the flask was removed to an ice bath to keep the reactants at a low temperature. 0.4363 g (2 mmole) of PMDA then was added in the flask and the solid content was 20 wt %. A concentrated poly(amic acid) (PAA) was progressively formed by stirring the reactants, and the concentrated PAA was further diluted with 2.3970 g of DMAc to obtain a PAA solution with 15 wt % solid content. After 2 hours stirring, the PAA solution was spread on a glass substrate and the thickness of the film was controlled within a range of 15˜45 micrometer (μm).

The glass substrate with the PAA film was placed in a circulator oven at 80° C. for 12 hours to remove the solvent in advance. Then, the glass substrate with the PAA film underwent thermal imidization by treating the PAA film with a temperature gradient from 100° C. to 300° C. for 3 hours. Finally, the treated glass substrate was immersed in water to separate the PI (polyimide) film and the glass substrate.

The molecular weight and solubility of the polyimides (5a˜5f) are shown in Table 1. Solubility of the polyimides were analyzed by dissolving the polyimides (5a˜5f) in different solvents, such as N-methyl-2-pyrrolidone (NMP), DMF, DMAc, dimethylsulfoxide (DMSO), and meta-Cresol (m-Cresol).

TABLE 1
The molecular weight and solubility of the polyimides (5a˜5f)

| | Number-average | Number-average | | | | | |
|---|---|---|---|---|---|---|---|
| | Molecular Weight | Molecular Weight | Solvent | | | | |
| Polyimides | (×10⁴) | (×10⁴) | NMP | m-Cresol | DMAc | DMSO | DMF |
| (5a) | 2.6 | 3.8 | +− | + | +− | +− | − |
| (5b) | 4.7 | 9.0 | − | − | − | − | − |
| (5c) | 7.0 | 12.5 | + | + | + | + | + |
| (5d) | 4.8 | 6.8 | − | − | − | − | − |
| (5e) | 8.0 | 14.6 | + | + | + | + | + |
| (5f) | 8.3 | 16.5 | + | + | + | + | + |

+: High solubility in solvent at room temperature.
+−: Low solubility in solvent at room temperature.
−: Insoluble in solvent at room temperature.

Referring to Table 1, the polyimides (c), (e), and (f, which have a number-average molecular weight in the range of 7.0˜8.3×10⁴ g/mole and a weight-average molecular weight in the range of 12.5˜16.5×10⁴ g/mole, were dissolved in DMF. For the low solubility polyimide, the low molecular weight portion were dissolved in DMF, so that the measured number-average molecular weight and weight-average molecular weight are less then the polyimides (c), (e), and (f. Table 1 shows that introducing the DOPO group would increase the solubility of the polyimides, so as to manufacturing the dissoluble polyimide.

The results of the different thermal analysis and thermogravimetry analyses of the polyimides (5a˜5f) are shown in Table 2.

TABLE 2
The molecular weight and solubility of the polyimides (5a˜5f)

| | | | | 5% mass loss | |
|---|---|---|---|---|---|
| | Glass transition | Tensile | | decomposition | Carbon |
| Poly- | temperature | Strength | Elongation | temperature | residue |
| imides | (° C.) | (MPa) | (%) | (° C.) | (%) |
| (5a) | 304 | 90 | 10.7 | 553 | 65 |
| (5b) | 266 | 87 | 7.5 | 572 | 65 |
| (5c) | 254 | 104 | 8.1 | 584 | 64 |
| (5d) | 277 | 87 | 8.5 | 597 | 64 |
| (5e) | 273 | 97 | 6.9 | 544 | 59 |
| (5f) | 230 | 85 | 8.9 | 566 | 62 |

Table 2 shows the polyimides (5a˜5f) exhibited a high glass transition temperature (Tg) that was within a range between about 230° C.˜304° C. The decomposition temperature (Td) at 5% mass loss of the polyimides (5a˜5f) were within a range between 544° C.˜597° C. The carbon residue was within a range between 59%˜64%. The phosphorous-containing thermoforming material of the present invention exhibits higher decomposition temperature than the phosphorous-containing polymers with P═O group in the main chain. The phosphorous-containing thermoforming materials, polyimides (5a˜5f), provide good mechanical properties such as about 90 MPa of tensile strength.

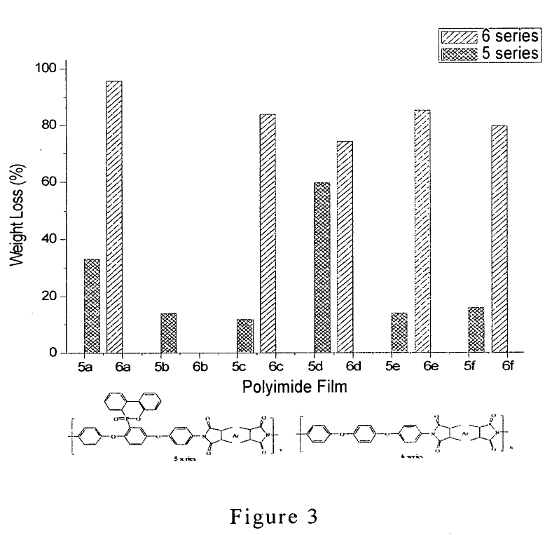

Referring to FIG. 3. FIG. 3 is a diagram of weight loss (%) of the polyimides (5a˜5f) in oxygen plasma destruction. The phosphorous-containing polyimides of the present invention have less weight loss (%) as compared with the phosphorous-free polyimides (6a˜6f). Poly(phosphate ester) can be formed by reacting the organic phosphorous with atomic oxygen to resist erosion from atomic oxygen attack. Therefore, the phosphorous-containing polyimides of the embodiments of the present invention provide the atomic oxygen resistance for Low-Earth orbit spacecraft applications.

### Preparation Example 4

Phosphorous-containing polyamides was prepared by reacting a DOPOBQ-AB with a series of diacids. The phosphorous-containing polyamides has a general formula are presented as follow:

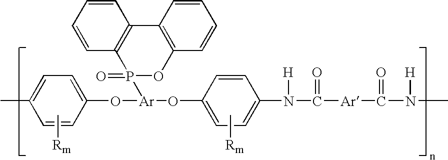

The “R” comprises hydrogen, —CH₃, —C₆H₅, or —CF₃. The “m” is an integer of 1˜2. The “Ar” is selected from the group consisting of following formulas:

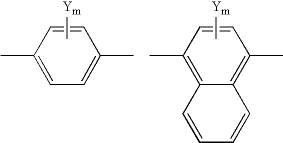

The “Y” comprises hydrogen, and C₁˜C₆ alkane. The “m” is an integer of 1˜2.

An exemplary synthesis strategy of the DOPO derived phosphorous-containing polyimides is shown in the following formula:

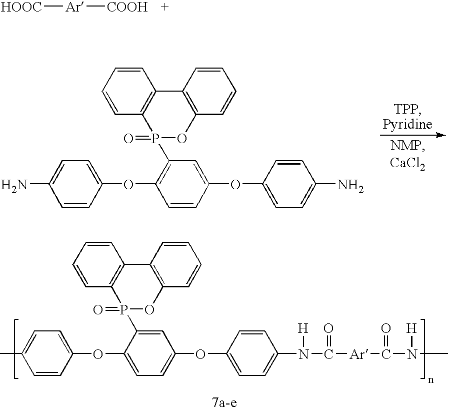

In accordance with embodiments of the present invention, the “Ar′” of diacids can be (a), (b), (c), (d), or (e) which are presented as follows:

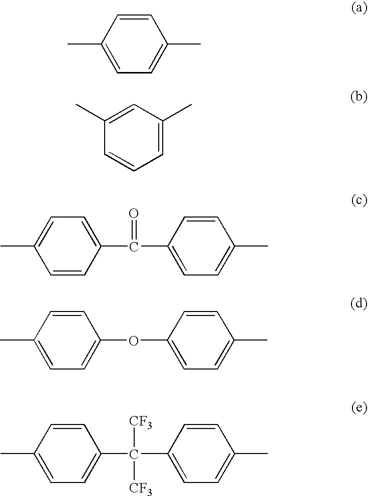

The synthesis of the DOPO derived phosphorous-containing polyamides (7a˜7f) may be accomplished by reacting the DOPOBQ-AB monomer with different diacids (a˜f) in a similar manner, an exemplary preparation process is stated in the following description. 0.6331 g (1.25 mmole) of DOPOBQ-AB, 0.2079 (1.25 mmole) g of terephthalic acid, 0.3 g calcium chloride (CaCl₂), 0.9 ml triphenyl phosphine (TPP), 1.2 ml pyridine and 5 ml NMP were stirred in a 100 ml 3-neck flask, and nitrogen was introduced into the 3-neck flask for 30 minutes. The reactants in the 3-neck flask were heated up to 100° C. for 4 hours to accomplish the reaction. Then the reactants in the 3-neck flask were cooled down to room temperature and titrated to 300 ml methanol to separate the precipitates. The precipitates were filtered and washed by methanol and hot water. The products were dried at 150° C. in an oven, and 0.7973 g polyamide (7a) was obtained.

The synthesized polyamide was added in a solvent (such as DMAc or NMP) to form a PA (polyamide) solution with 20 wt % solid content. The PA solution was spread on a glass substrate and the thickness of the film was about 45 micrometer (μm). The glass substrate with the PA film was placed in a circulator oven at 80° C. for 12 hours to remove the solvent in advance. Then, the glass substrate with the PA film was treated at 200° C. for 2 hours. Finally, the treated glass substrate was immersed in water to separate the PA (polyamide) film and the glass substrate.

The molecular weight and solubility of the polyimides (7a˜7e) are shown in Table 3. Solubility of the polyimides were analyzed by dissolving the polyimides (7a˜7e) in different solvents, such as N-methyl-2-pyrrolidone (NMP), DMF, DMAc, dimethylsulfoxide (DMSO), and meta-Cresol (m-Cresol).

TABLE 3
The molecular weight and solubility of the polyimides (57a˜7e)

| | Number-average | Number-average | | | | | |
|---|---|---|---|---|---|---|---|
| | Molecular Weight | Molecular Weight | Solvent | | | | |
| Polyamides | (×10⁴) | (×10⁴) | NMP | m-Cresol | DMAc | DMSO | DMF |
| (7a) | 9.9 | 24.8 | + | + | + | + | + |
| (7b) | 4.2 | 7.5 | + | + | + | + | + |
| (7c) | 21.3 | 28.4 | +− | − | − | − | +− |
| (7d) | 6.7 | 12.7 | + | + | + | + | + |
| (7e) | 10.5 | 28.2 | + | + | + | + | + |

+: High solubility in solvent at room temperature.
+−: Low solubility in solvent at room temperature.
−: Insoluble in solvent at room temperature.

Referring to Table 3, the number-average molecular weight of the polyamides (a)˜(e) were between a range of 4.2˜21.3×10⁴ g/mole and the weight-average molecular weight between a range of 7.5˜28.4×10⁴ g/mole, were dissolved in DMF. Table 3 has proved that introducing the DOPO group would increase the solubility of the polyamides, so as to manufacture the dissoluble polyamide.

The results of the different thermal analysis and thermogravimetry analysis of the polyimides (7a˜7e) are shown in Table 4.

TABLE 4
The molecular weight and solubility of the polyamides (7a˜7e)

| | | | | 5% mass loss | |
|---|---|---|---|---|---|
| | Glass transition | Tensile | | decomposition | Carbon |
| Poly- | temperature | Strength | Elongation | temperature | residue |
| amides | (° C.) | (MPa) | (%) | (° C.) | (%) |
| (7a) | 239 | 86 | 9.7 | 533 | 68 |
| (7b) | 209 | 83 | 8.9 | 507 | 63 |
| (7c) | 260 | 94 | 7.6 | 508 | 68 |
| (7d) | 232 | 81 | 9.4 | 514 | 65 |
| (7e) | 256 | 91 | 5.7 | 525 | 53 |

Table 4 shows the polyimides (5a˜5f) exhibited high glass transition temperature (Tg) within a range between about 209° C.˜259° C. The decomposition temperature (Td) at 10% mass loss of the polyimides (5a˜5f) were within a range between 507° C.˜533° C. The carbon residue was within a range between 63%˜68%. The phosphorous-containing polyamides (7a˜7e), provide good mechanical properties.

In conclusion, both the polyimides and the polyamides of the embodiment of the present invention are solvent-soluble, with high glass transition temperature, and oxygen plasma resistances. The polyimides and the polyamides of the embodiment of the present invention are applied to Low-Earth orbit application and potential materials for making FPC broad.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A process of manufacturing DOPO derivatives for a printed circuit board and Low-Earth Orbit (LEO) spacecraft applications, comprising:
reacting a DOPO derivative with 1-fluoro-4-nitrobenzene, wherein the DOPO derivative is selected from the group consisting of DOPOBQ and DOPONQ, to form a phosphorus containing aromatic dinitro-compound DOPOBQ-NB or DOPONQ-NB; and
synthesizing a DOPO derivative for a printed circuit board and Low-Earth Orbit (LEO) spacecraft applications by using the one of the DOPOBQ-NB and DOPONQ-NB as a initiating monomer.

2. The process of claim 1, wherein the phosphorus containing aromatic dinitro-compound DOPOBQ-NB have a structure represented by a following formula 1:

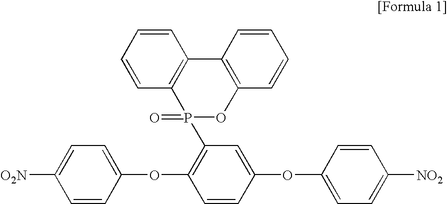

3. The process of claim 2, further comprises reacting the DOPOBQ-NB with a substituent group containing p-halo nitrobenzene to synthesize a DOPO derivative has a structure represented by a following formula 2: 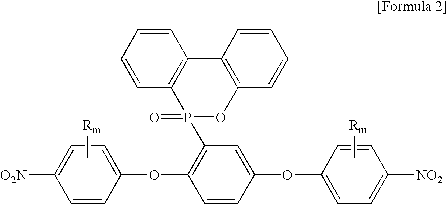
wherein “R” comprises hydrogen, —CH₃, —C₆H₅, or —CF₃, and “m” is an integer of 1˜2.

4. The process of claim 3, wherein the substituent group containing p-halo nitrobenzene has a general formula represented by a following formula 3: 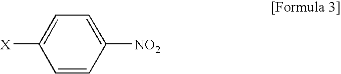
wherein the “X” is selected from the group consisting of F, Cl, Br, and I.

5. The process of claim 3, further comprises reacting the DOPO derivative represented by the formula 2 with hydrogen to synthesize a DOPO derivative has a general formula represented by a following formula 4: 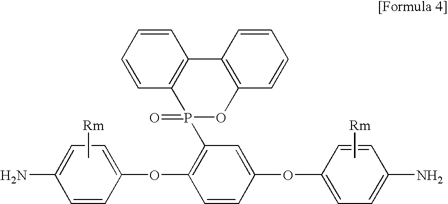
wherein “R” comprises hydrogen, —CH₃, —C₆H₅, or —CF₃, and “m” is an integer of 1˜2.

6. The process of claim 2, further comprises reacting the DOPOBQ-NB with hydrogen to synthesize a DOPO derivative DOPOBQ-AB has a structure represented by a following formula 5:

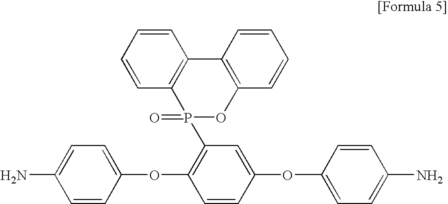

7. The process of claim 6, further comprises reacting the DOPOBQ-AB with various dianhydrides to synthesize DOPO derivatives has a general formula represented by the following formula 6: 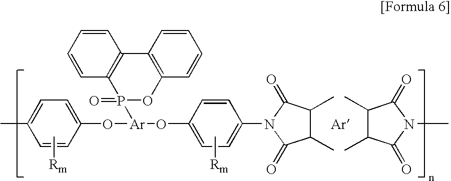
wherein “R” comprises hydrogen, —CH₃, —C₆H₅, or —CF₃, and “m” is an integer of 1˜2.

8. The process of claim 7, wherein the dianhydrides has a general formula represented by a following formula 7:

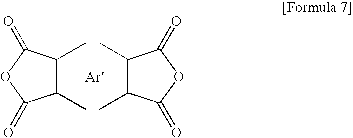

9. The process of claim 8, wherein the “Ar′” is selected from the group consisting of following formulas (a), (b), (c), (d), (e), and (f):

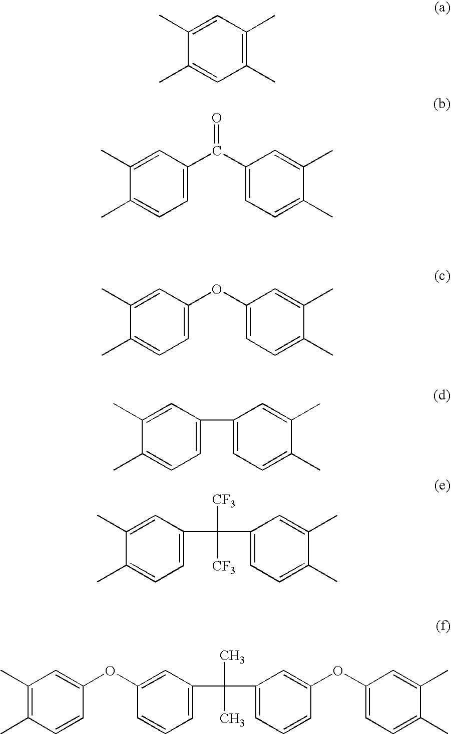

10. The process of claim 7, wherein the “Ar” is selected from the group consisting of following formulas (g), and (h): 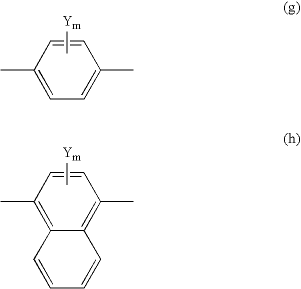
wherein “Y” comprises hydrogen, and C₁˜C₆ alkane, and “m” is an integer of 1˜2.

11. The process of claim 6, further comprises reacting the DOPOBQ-AB with various diacids to synthesize DOPO derivatives has a general formula represented by the following formula 8: 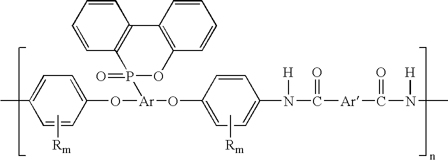
wherein “R” comprises hydrogen, —CH₃, —C₆H₅, or —CF₃, and “m” is an integer of 1˜2.

12. The process of claim 11, wherein the diacids has a general formula represented by a following formula:
HOOC—Ar′—COOH

13. The process of claim 12, wherein the “Ar′” is selected from the group consisting of following formulas (a), (b), (c), (d), and (e):

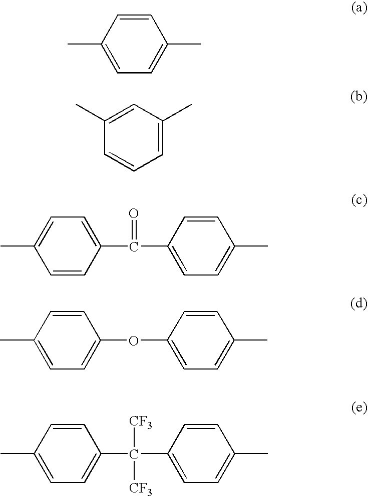

14. The process of claim 11, wherein the “Ar” is selected from the group consisting of following formulas (f), and (g): 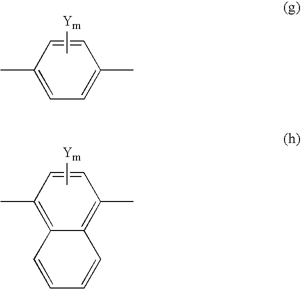
wherein “Y” comprises hydrogen, and C₁˜C₆ alkane, and “m” is an integer of 1˜2.

15. The process of claim 1, wherein the phosphorus containing aromatic dinitro-compound DOPONQ-NB have a structure represented by a following formula 9:

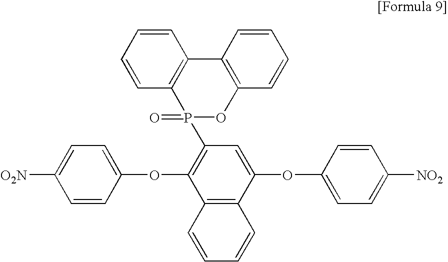

16. The process of claim 15, further comprises reacting the DOPONQ-NB with a substituent group containing p-halo nitrobenzene to synthesize a DOPO derivative has a structure represented by formula 10: 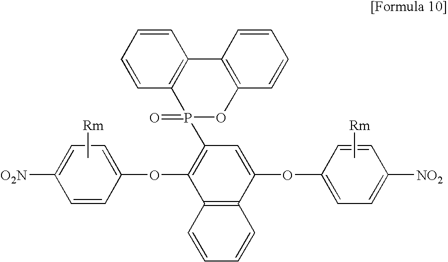
wherein “R” comprises hydrogen, —CH₃, —C₆H₅, or —CF₃, and “m” is an integer of 1˜2.

17. The process of claim 16, wherein the substituent group containing p-halo nitrobenzene has a general formula represented by following formula: 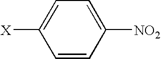
wherein the “X” is selected from the group consisting of F, Cl, Br, and I.

18. The process of claim 15, further comprises reacting the DOPO derivative represented by the formula 10 with hydrogen to synthesize a DOPO derivative has a general formula represented by a following formula 11: 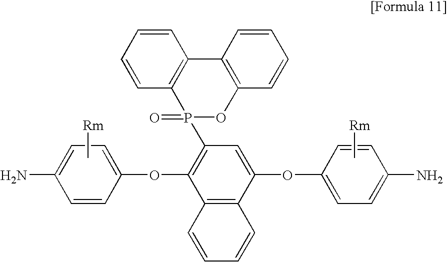
wherein “R” comprises hydrogen, —CH₃, —C₆H₅, or —CF₃, and “m” is an integer of 1˜2.

19. The process of claim 15, further comprises reacting the DOPONQ-NB with hydrogen to synthesize a DOPO derivative DOPONQ-AB has a structure represented by the following formula 12:

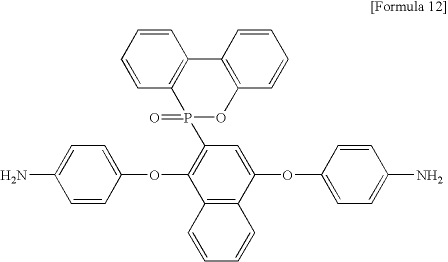

20. The process of claim 1, wherein the phosphorus containing aromatic dinitro-compound is synthesized by adding CsF, KF, CsCl, KCl, K₂CO₃, Na2CO3, KOH or NaOH as a catalyst.
